(19) United States
(12) Patent Application Publication
(10) Pub. No.: US 2024/0156744 A1
(43) Pub. Date: May 16, 2024

(54) METHOD OF APPLYING ELECTRICAL IMPULSES FOR THE PURPOSE OF LOADING VARIOUS MOLECULES INTO PLANT-DERIVED NANOVESICLES

(71) Applicant: EXO LAB ITALIA, S.r.l., Pescara (IT)

(72) Inventors: Rossella DI RAIMO, Pescara (IT); Davide MIZZONI, Pescara (IT); Mariantonia LOGOZZI, Pescara (IT); Stefano FAIS, Pescara (IT); Enrico Pierluigi SPUGNINI, Pescara (IT); Alfonso BALDI, Pescara (IT); Cosimo ASSUMMA, Pescara (IT)

(73) Assignee: EXO LAB ITALIA, S.r.l., Pescara (IT)

(21) Appl. No.: 18/282,465

(22) PCT Filed: Mar. 23, 2022
(86) PCT No.: PCT/IB2022/052663

§ 371 Date: Sep. 15, 2023

(30) Foreign Application Priority Data
Mar. 25, 2021 (IT) 102021000007304

(51) Int. Cl. A61K 9/51; A61K 8/14; A61K 9/00; A61K 45/06

(52) U.S. Cl. CPC A61K 9/5176; A61K 8/14; A61K 9/0009; A61K 9/5192; A61K 45/06; A61K 2800/83

(57) ABSTRACT

Method for loading various molecules into plant-derived nanovesicles, comprising the following steps: a. suspending the isolated nanovesicles in a phosphate buffered saline; b. analyzing the suspended nanovesicles with a technique called “Nanoparticle Tracking Analysis” using a “Nanosight” for the evaluation of concentration and size distribution; c. re-suspending the nanovesicles in phosphate buffered saline; d. transferring the nanovesicles to sterile means; e. adding the fluorescent chemical to be loaded; f. treating the nanovesicles in the sterile means to facilitate the entry of the molecule to be loaded through their cell membrane; g. transferring the nanovesicles loaded with the desired molecule into ultra¬centrifuge tubes; h. re-suspending the pellet containing the nanovesicles in phosphate buffered saline and storing the supernatant obtained from the ultracentrifugation as a control for subsequent analysis; i. re-suspending the nanovesicles in phosphate buffered saline and proceed with testing.

### BACKGROUND OF THE INVENTION

### 1. Field of the Invention

The present invention relates to an innovative method for loading various molecules into plant-derived nanovesicles. In particular, the nanovesicles are extracted from organic plants and the method, which is another object of the present invention, makes possible to load into nanovesicles substances of various kinds, for example: vitamins, substances for cosmetic use, drugs and agents for medical, veterinary, pharmacological and food use.

### 2. Brief Description of the Prior Art

As is well known, the use of nanostructures capable of transporting molecules of various kinds to their site of action and facilitating and improving their effectiveness is one of the main areas of investment in the biotechnology industry. Until now, the idea has been to artificially construct “lipid rafts” that could encapsulate the molecule and thus protect it until it reached its intended site of action. These lipid structures have been called ‘liposomes’ and are, to date, the only device already tested in clinical trials.

The results obtained have not been encouraging due to a number of problems linked to the artificial nature of liposomes, which have favored systemic toxicity rather than the expected increase in efficacy. A number of publications by the same writer have demonstrated the ability of natural nanovesicles, called exosomes, to transport molecules of various kinds, from drugs (Federici C. et al 2014, lessi et al 2016) to nanomaterials (Logozzi et al 2020). It was therefore decided to experiment with various methods in order to facilitate the entry and permanence of molecules of various natures within exosomes. To go into more detail, unlike exosomes, which are produced naturally, liposomes are produced synthetically and have a lipid bilayer structure.

Liposomes are designed to allow both the encapsulation of hydrophilic molecules, such as siRNA, DNA and RNA, in the aqueous core of the vesicle, and hydrophobic bioactive compounds, such as proteins, peptides, phenolics and antibodies, in the lipid bilayer (Qaada et al., 2014).

As is known in the state of the art, liposomes are prepared by membrane extrusion, sonication, microemulsification and freeze-thawing and have been used in the “delivery” of many drugs, for example in the formulation of amphotericin B, doxorubicin, verteporfin, cytarabine, morphine sulphate and daunorubicin (Fan and Zhang, 2013). The preparation of liposomes, however, can be problematic as it requires numerous treatments and chemical steps to modify the structure of the lipid bilayer using proteins, ligands and antibodies (Ishida et al., 1999; Theek et al., 2016). In addition, the clinical use of liposomes has demonstrated a high level of toxicity, due to the fact that most liposomes enter the body's catabolic system, being largely sequestered in catheretic organs such as spleen and liver; this obviously, in addition to a high level of toxicity, leads to a very low level of efficiency.

Information on plant-derived nanovesicles is generating much interest in their possible use as molecule transporters for therapeutic purposes. Indeed, natural (‘exosomes-like’) nanovesicles and liposomes have similar physico-chemical properties, but unlike liposomes, plant-derived exosomes do not enter the catabolic system of the body's filter organs. In addition, the inherent biochemical similarities between the surface of the exosome and that of the original plants make it more feasible, compared to liposomes, to direct exosomes to a specific target (Keller et al., 2006).

Furthermore, the industrial use of plant-derived nanovesicles is bio-renewable and sustainable. These observations suggest that the use of plants as an industrial source (nanofactories) for the manufacture of nanoparticles for preventive and therapeutic use could represent a new approach in nanomedicine. Indeed, it is expected that the promotion of research and development of nanovesicles for the transport of therapeutic molecules will contribute significantly to the development of natural nanomedicines. Thus, nanovesicles of plant origin have all the characteristics to allow adequate transport of therapeutic molecules with a low level of toxicity, and not least with a very low level of environmental pollution.

However, the purification and concentration of plant-derived nanovesicles involves a totally different production process from the synthetic liposome process.

There is no known efficient method for loading molecules of various kinds into nanovesicles of plant origin.

Electroporation, for example, is a known technique, commonly applied to skin or tissue, i.e. to cells in general, to facilitate the entry of molecules into them. Passive diffusion is also a known method of moving chemicals across biological membranes by simple diffusion.

However, there are no known methods of application to nanovesicles, and even less so to plant-derived nanovesicles, to facilitate the entry of the molecule to be loaded through their cell membrane.

A technical problem that has not yet been overcome is related to the possibility of allowing satisfactory ‘up-take’ of the molecules of interest within intact nanostructures such as plant-derived nanovesicles, while maintaining the integrity of the structures themselves.

### SUMMARY OF THE INVENTION

Thus, an object of the present invention is a method for loading various molecules into nanovesicles extracted from biological plants. In particular, the method makes it possible to satisfactorily load plant-derived nanovesicles, by means of techniques for facilitating the entry of the molecule to be loaded through the cell membrane of the nanovesicles themselves.

In particular, the method may comprise a loading step by electroporation or a loading step by passive diffusion, for the diffusion inside the nanovesicles of, for example, vitamins, substances for cosmetic use, drugs or agents for medical, veterinary pharmacological and food use.

The method object of the present invention provides, according to a first form of implementation, the use of an apparatus for loading nanovesicles by a physicochemical method, through transient destabilization of said nanovesicles (electroporation). According to this methodology, combinations of single electrical pulses and as a wave train are used, at voltages varying between 20 and 400 volts, and of varying duration, in order to use the most efficient sequence for loading. The applied wave trains induce a transient permeabilization of the nanovesicle or exosome, which will return to the initial stage of electrical inertia after a variable time of less than thirty minutes.

In an alternative embodiment, the method according to the invention comprises a treatment of the nanovesicles that allows loading by passive diffusion.

Advantageously, such a permealization period will lead to increased transmembrane trafficking which will allow exosomal loading with various molecules depending on the required purpose.

Advantageously, electrical and chemical-physical parameters (e.g. pH) are constantly measured during permeabilisation and loading in order to improve the efficiency of the procedure.

The nanovesicles extracted from organic plants, are obtained through a methodology for which a parallel patent application has been applied for by the writer, which consists of serial centrifugations and ultracentrifugations, as required by internationally shared standard procedures (Thery C. et al. J Extracell Vesicles.

2018; 7(1): 1535750). Advantageously, the nanovesicles were extracted from juice squeezed from organic vegetables, such as Citrus paradisi, Citrus Lemon (L.), Citrus Reticulata, Citrus Bergamia, Actinidia Chinensis, Mangifera Indica, Carica Papaya Linn, Citrus Sinensis, Malus domestica.

Accordingly, according to the present invention, a novel method is defined for loading molecules of various kinds into plant-derived nanovesicles, as specified in the appended independent claim.

The dependent claims outline particular and further advantageous aspects of the invention.

### BRIEF DESCRIPTION OF THE DRAWINGS

These and other advantages of the invention will now be described in detail, with reference to the accompanying drawings, which represent an exemplary embodiment of the invention, in which:

FIG. 1 shows a graph of the loading, by the method of the present invention, of Acridine Orange (AO) at increasing concentrations into an increasing number of nanovesicles isolated from an organic fruit mix;

FIG. 2 shows the graph of loading, by the method of the present invention, of Acridine Orange (AO) into nanovesicles isolated from a mixture of organic fruits and comparison with commercial liposomes;

FIG. 3 shows the graph of loading, by the method of the present invention, of vinblastine (VBL) into nanovesicles isolated from a mixture of organic fruits and comparison with commercial liposomes;

FIG. 4 shows the graph of loading, by the method of the present invention, of an antibody bound to a probe (AlexaFluor 488) into nanovesicles isolated from a mix of organic fruit and comparison with commercial liposomes;

FIG. 5 shows Nanosight analysis of nanovesicles isolated from a mix of organic fruit according to the method of the present invention for Acridine Orange (AO) loading;

FIG. 6 shows the graph of the intensity of the refractive index of Acridine Orange (AO) in Nanovesicles isolated from a mixture of organic fruits according to the method object of the present invention;

FIG. 7 shows fluorescence microscopy images of the effect of Acridine Orange treatment loaded with nanovesicles extracted from an organic fruit mix on human melanoma cells and comparison with commercial liposomes;

FIG. 8 shows the graph of the evaluation of cytotoxicity on leukaemia cell line (CEM) and drug-resistant leukemia cell line (CEM/VBL100) by nanovesicles isolated from a mix of organic fruit and loaded according to the method of the present invention;

FIG. 9 shows the graph of the evaluation of cytotoxicity on the human melanoma cell line by nanovesicles isolated from a mixture of organic fruits and loaded with Acridine Orange, according to the method of the present invention and the comparison with Acridine Orange alone and with commercial liposomes;

FIG. 10 shows the graph of the stability tests of the retention of the drug loaded in the nanovesicles isolated from a mixture of organic fruits, according to the method of the present invention;

FIG. 11 shows the graph of the impedance of the nanovesicles extracted from organic lemon juice compared to the impedance recorded in the liposomes,

FIG. 12 shows the graph of the passive loading, by the method of the present invention, of the fluorescent dye: 5(6)-Carboxyfluorescein diacetate N-succinimidyl ester (CFSE) through buffers with different ionic strengths from the starting nanovesicles, isolated from a mixture of biological fruits;

FIG. 13 shows the graph of passive loading, by the method of the present invention, of vinblastine (VBL) through buffers with ionic strengths different from the starting nanovesicles, isolated from juice extracted from organic pink grapefruits and the comparison with exosomes of macrophages of human origin.

### DETAILED DESCRIPTION OF THE INVENTION

According to the present invention, the nanovesicles used by the method object of the present invention are extracted from organic plants, i.e. without the use of chemical pesticides, synthetic fertilizers, antibiotics and other substances which are subject to strict restrictions.

The attached figures show the results of tests carried out on nanovesicles loaded by the innovative method of the present invention.

The method for loading molecules of various nature into nanovesicles of plant origin comprises the following steps:
the isolated nanovesicles (from 1×10⁶ to 1×10¹²) were suspended in a 1× phosphate-buffered saline (PBS) at pH 7.4, without calcium and magnesium; PBS is an aqueous saline solution containing sodium chloride, sodium phosphate and potassium chloride;
the nanovesicles re-suspended in 1×PBS were analysed by Nanoparticle Tracking Analysis using Nanosight for the evaluation of concentration and size distribution;
from 10⁶ to 10¹³ nanovesicles re-suspended in 400 to 1000 pi of PBS 1× were transferred into sterile 0.4 cm cuvettes by electroporation and the fluorescent chemical to be loaded (e.g. Acridine Orange) was added at concentrations ranging from 0.1 to 100 pg/mL;
the cuvettes containing the nanovesicles and the compound to be charged were subjected to electroporation by means of an electroporator, using the following pulse trains:

300 V with eight pulses
300 V with a first pulse
a series of eight pulses of 20 V, or 50 V, or 80 V, or 100 V;
the nanovesicles loaded with the desired molecule were transferred into ultracentrifuge tubes and subjected to ultracentrifugation at 110,000 g for ninety minutes to remove the compound that did not enter;
the pellet containing the nanovesicles was resuspended in PBS (50 to 1000 pi) and the supernatant obtained from the ultracentrifugation was kept as a control for later analysis;
the nanovesicles are resuspended in 1×PBS for testing.

The voltages used in the present method were derived on the basis of experience with the transport of molecules inside cells, taking into account the smaller size and different stability of the exosomal membrane. The pulses were generated with the following function: the first pulse serves to destabilize the membrane by making it permeable, the next pulse train serves to facilitate transmembrane ionic (or molecular) transit.

Alternatively, the method for loading molecules of various nature inside plant-derived nanovesicles, according to the present invention, is characterised by a treatment of the nanovesicles that allows loading by passive diffusion. The method comprises the following steps:
isolated nanovesicles (from 1×10⁹ to 1×10¹⁰) were suspended in phosphate buffered saline 1× (PBS, Phosphate Buffered Saline) to pH 7.4, without calcium and magnesium; PBS is an aqueous saline solution containing sodium chloride, sodium phosphate and potassium chloride;
nanovesicles re-suspended in 1×PBS were analysed by Nanoparticle Tracking Analysis using Nanosight to assess concentration and size distribution;
from 10⁹ to 10¹⁰ Nanovesicles re-suspended in 1000 pi of 1×PBS were incubated with the fluorescent chemical CFSE to be loaded at concentrations from 1 to 10 mM and with the fluorescent chemical VBL at concentrations from 0.1 to 0.5 pg/mL;
the tubes containing the nanovesicles and the compound to be loaded were incubated at optimal times and temperatures to facilitate loading, namely 30 min at 37° C. for CFSE loading and 2 h at room temperature (T amb);
the nanovesicles loaded with the desired molecule were transferred into ultracentrifuge tubes and subjected to ultracentrifugation at 110,000 g for ninety minutes to remove the compound that did not enter;
the pellet containing the nanovesicles was re-suspended in PBS (50 to 1000 ml) and the supernatant obtained from the ultracentrifugation was kept as a control for later analysis;
the nanovesicles are re-suspended in 1×PBS for testing.

Advantageously, simple (or passive) diffusion allows the desired molecule to be loaded without the aid of instrumentation or energy to facilitate the entry of the compound through the membrane of the nanovesicles, but exclusively according to a concentration gradient. Obviously, this loading process depends on several factors, especially the size (very small), hydrophobic properties and chemical structure of the molecule to be loaded, as well as the buffer used for loading.

The instruments used for testing are:
the spectrofluorimeter, for evaluation of the loading of the fluorescent compound obtained following electroporation. The loading of Acridine Orange into the exosomes was evaluated by spectrofluorimeter as fluorescence emission at 535 nm following excitation at 485 nm. The final concentrations loaded into the plant nanovesicles were calculated by constructing a calibration curve obtained by reading the fluorescence emitted by the drug or antibody in the same experiment;
Nanosight (Nanoparticle Tracking Analysis), for the evaluation of the concentration, size distribution and refractive index of the loaded nanovesicles (quality control).

The same steps of the method described above were used to load in commercial liposomes (from 10⁶ to 10¹³ liposomes) the compound (e.g. Acridine Orange,) at concentrations from 0 to 100 pg/Ml, to be used as a control of loading and effect on target cells.

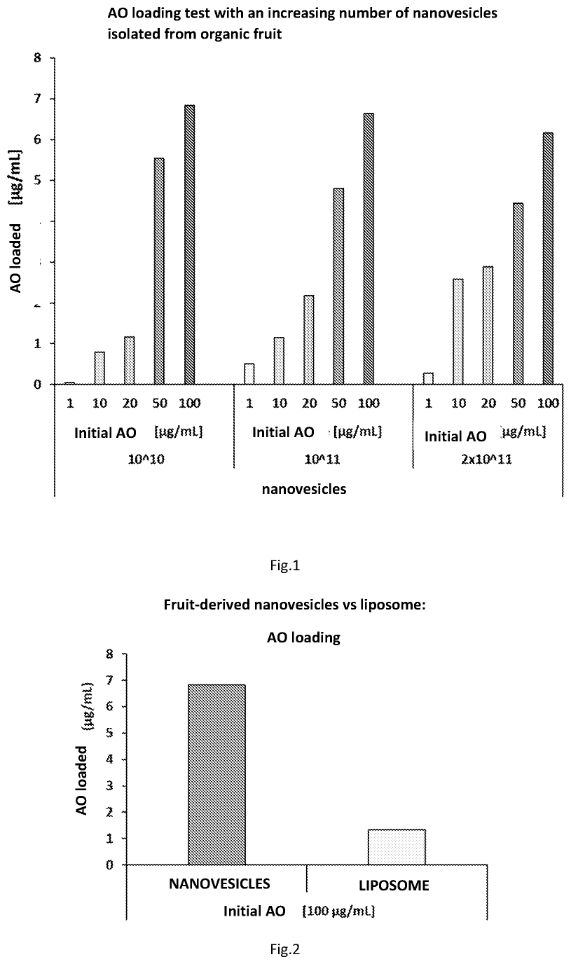

The tests performed on the nanovesicles treated according to the method are shown in the attached FIGS. 1-11.

In particular, FIG. 1 shows loading tests using the method described in the present invention of Acridine Orange (AO) at increasing concentrations in an increasing number of nanovesicles isolated from an organic fruit mix. Increasing amounts of nanovesicles (from 10¹⁰ to 2×10¹¹) isolated from a mixture of organic fruits (see experimental protocol) were charged, by means of the same electroporation pulse (300 V with the first biphasic pulse followed by a series of pulses, from 20V to 100 V), with increasing concentrations of AO (from 1 to 100 pg/mL). The results depicted in FIG. 1 show that the plant nanovesicles are able to load a progressively higher AO concentration as the AO dose and the number of nanovesicles increase until saturation is reached.

FIG. 2 shows loading, according to the method described in the present invention, of Acridine Orange (AO) into nanovesicles isolated from an organic fruit mix: comparison with commercial liposomes. The same number of nanovesicles (from 10⁶ to 10¹¹) isolated from a mixture of organic fruit (see experimental protocol) and commercial liposomes was loaded, using the same electroporation pulse (300 V with the first biphasic pulse followed by a series of pulses, from 20 V to 100 V), with an initial concentration of 100 pg/mL of AO. The results shown in FIG. 2 show that the plant nanovesicles manage to load a significantly higher concentration of OA (>5 times) than liposomes.

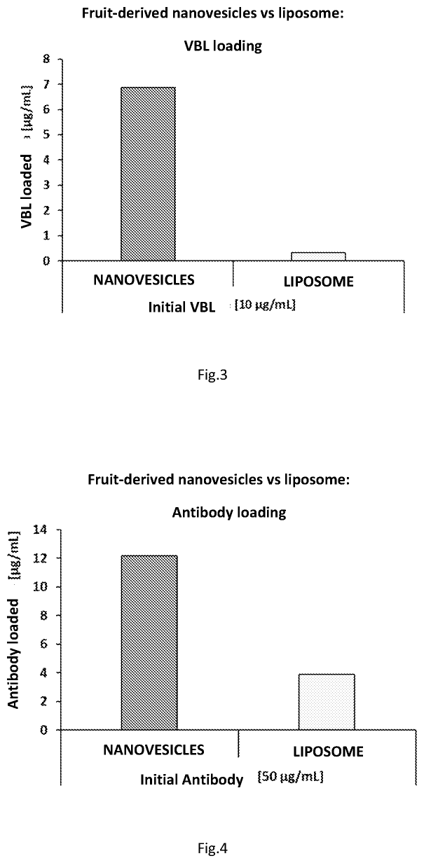

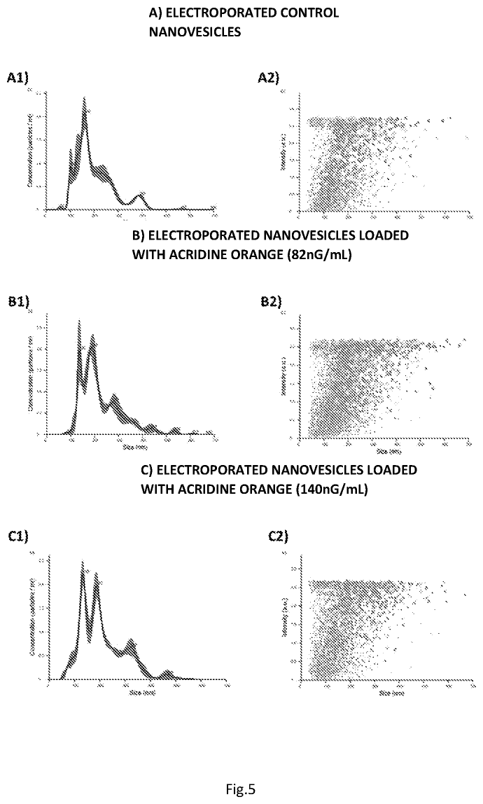

FIG. 3 shows loading according to the method described in the present invention, of vinblastine (VBL) into nanovesicles isolated from an organic fruit mix and comparison with commercial liposomes. The same number of nanovesicles (106 to 1011) isolated from a mixture of organic fruit and commercial liposomes were loaded, by means of the same electroporation pulse (300 V with 1st biphasic pulse followed by a series of pulses, from 20V to 100 V), with an initial concentration of 10 pg/mL of VBL. The results shown in the figure show that the plant nanovesicles manage to load a significantly higher concentration of AO (21-fold) than liposomes.

FIG. 4 shows loading according to the method described in the present invention, of an antibody bound to a probe (AlexaFluor 488) into nanovesicles isolated from an organic fruit mix and comparison with commercial liposomes.

The same number of nanovesicles (from 10⁶ to 10¹¹) isolated from a mixture of organic fruit and commercial liposomes was loaded, using the same electroporation pulse (300 V with the first biphasic pulse followed by a series of pulses, from 20 V to 100 V), with an initial concentration of 50 pg/mL of an Antibody bound to a fluorescent probe (AlexaFluor 488). The results shown in the figure show that plant nanovesicles are able to load a significantly higher concentration of Antibody (3 times) than liposomes.

Advantageously, the loading of Acridine Orange, Vinblastine and Antibody into the exosomes was evaluated in the spectrofluorimeter as fluorescence emission at 535 nm following excitation at 485 nm. The final concentrations loaded into the plant nanovesicles were calculated by constructing a calibration curve obtained by reading the fluorescence emitted by the drug or antibody in the same experiment. FIG. 5 shows Nanosight analysis of Nanovesicles isolated from a mix of organic fruit according to the method of the present invention, for AO loading. The same number of nanovesicles (106 to 1011) isolated from a mixture of organic fruit and commercial liposomes were loaded, by means of the same electroporation pulse (300 V with 1st biphasic pulse followed by a series of pulses, from 20V to 100 V), with a concentration of 82 ng/mL and 140 ng/mL of AO. The nanovesicles were checked by Nanosight for size distribution, concentration and refractive index. Loading with AO induced a double-peaked distribution of the plant nanovesicles (B1 and C1), in contrast to the control nanovesicles (A1) whose distribution is more uniform. The refractive indices of the drug-loaded nanovesicles (B2 and C2) show a slight decrease in intensity compared to the control nanovesicles (A2).

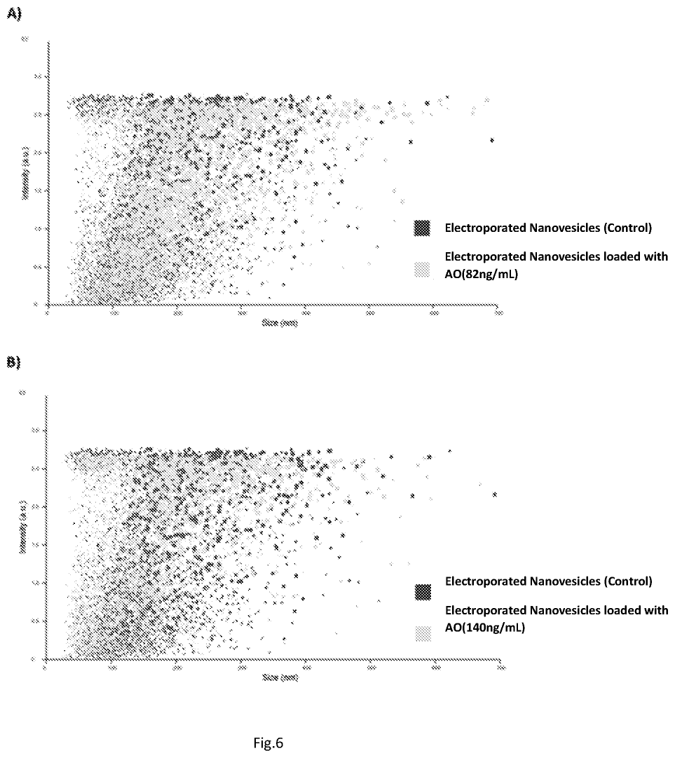

FIG. 6 shows the loading, according to the method of the present invention, of AO into nanovesicles isolated from an organic fruit mix induces a decrease in the intensity of the refractive index. In (A) and (B), the refractive indices between control nanovesicles and nanovesicles loaded with an AO concentration of 82 ng/mL (A) and 140 ng/mL (B), respectively, are shown. The intensity of the drug-loaded nanovesicles decreases compared to their control.

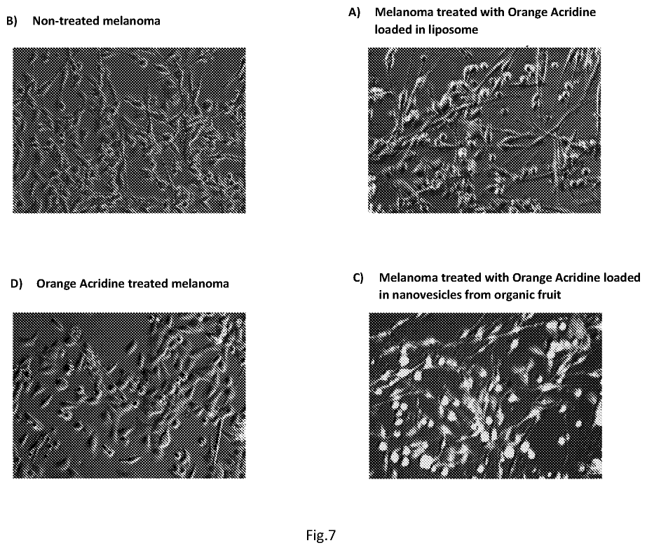

FIG. 7 shows the effect of Acridine Orange treatment loaded in nanovesicles extracted from a biological fruit mix on human melanoma cells and the comparison with commercial liposomes. The same number of nanovesicles (from 10⁶ to 10¹¹) isolated from a mixture of organic fruit and commercial liposomes was loaded with AO [200 ng/mL] using the same electroporation pulse (300 V with first biphasic pulse followed by a series of pulses from 20 V to 100 V). The melanoma cells were treated and the up-take of AO was assessed by inverted fluorescence microscopy. The results shown in the figure show that the plant nanovesicles are able to transfer a significantly higher concentration of AO (higher fluorescence) than liposomes and acridine orange alone.

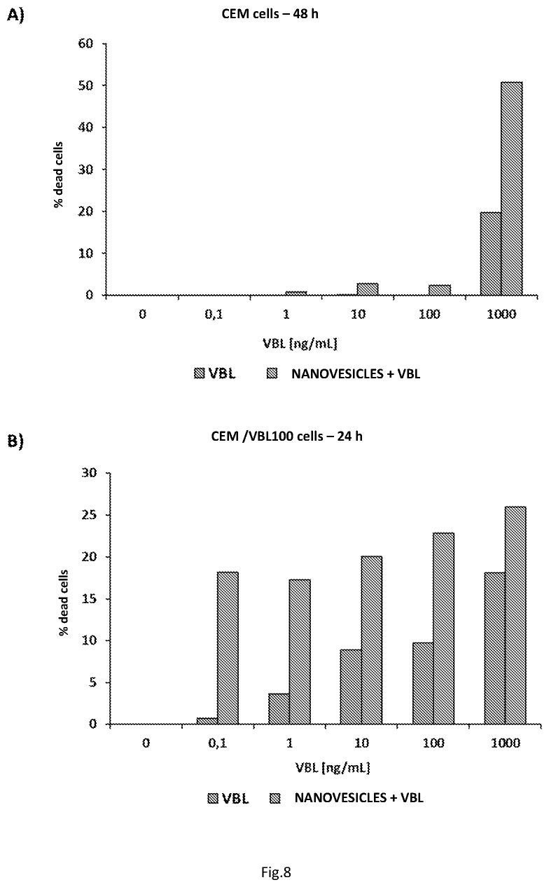

FIG. 8 shows the graph of the evaluation of cytotoxicity on the OEM leukaemic cell line and the CEM/VBL100 drug-resistant leukaemic cell line by nanovesicles isolated from an organic fruit mix and loaded by electroporation with VBL. Increasing concentrations of VBL alone and loaded into the plant nanovesicles were used to assess the cytotoxic effect on CEM and CEM/VBL100 leukaemic cells by Trypan blue assay after 48 and 24 h of incubation, respectively. A) The cytotoxic effect on CEM cells starts to be evident at a concentration of 1000 ng/mL and is enhanced in VBL loaded in the plant nanovesicles (2.6-fold higher than in VBL alone). B) The cytotoxic effect on drug-resistant CEM/VBL100 cells begins to be evident (18.1% mortality) at low concentrations of VBL (0.1 ng/mL) when the drug is loaded into the plant nanovesicles, which is 26-fold higher than VBL 0.1 ng/mL alone, which induces almost no mortality (0.7%).

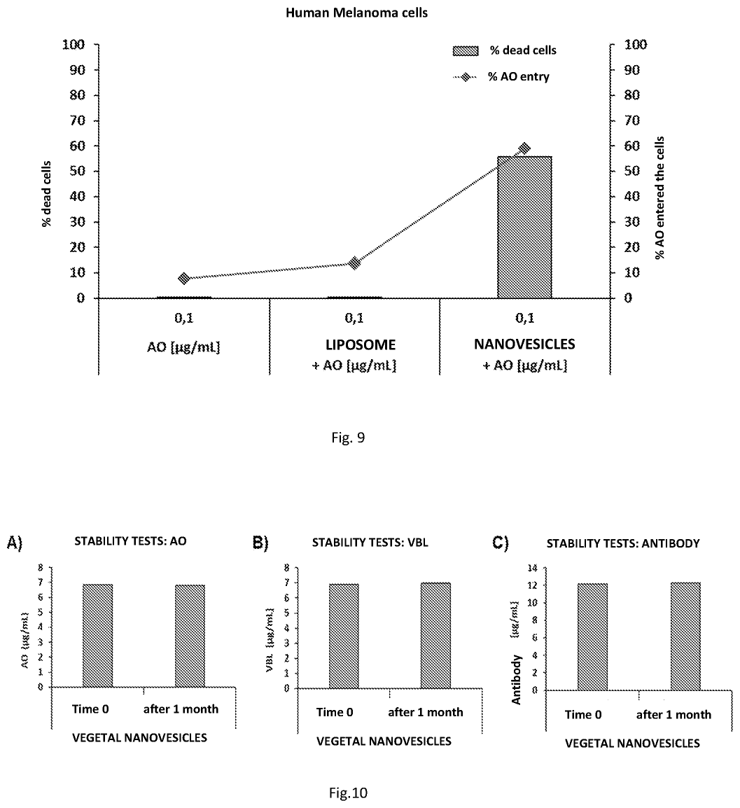

FIG. 9 shows the evaluation of cytotoxicity on the human melanoma cell line by nanovesicles isolated from an organic fruit mix and loaded by the method of the present invention with AO and comparison with acridine orange alone and with commercial liposomes. Human melanoma cells cultured in culture medium of different ionic strength were treated with a concentration of AO of 0.1 pg/mL alone and loaded in the same number of fruit-isolated nanovesicles and liposomes. Acridine orange was not activated with blue light. The cytotoxic effect was evident (55.7%) mainly on cells grown in unbuffered medium when AO is loaded into fruit nanovesicles, whereas no cytotoxic effect was detected at the same concentration of AO (0.1 pg/mL) when the drug is used alone or in combination with liposomes. The percentage of AO transferred to tumour cells was significantly higher (59%) when AO is loaded into fruit nanovesicles and the cells are cultured in unbuffered medium. The amount of AO able to enter melanoma cells was significantly reduced when the drug was used alone or in combination with liposomes.

FIG. 10 shows stability tests of the retention of the drug loaded by the method of the present invention in nanovesicles isolated from an organic fruit mix. Stability tests of the drug (AO and VBL) and antibody (AlexaFluor488) loaded into the plant nanovesicles were conducted one week and one month after loading by electroporation. The results obtained by fluorescence reading on the spectrofluorometer indicated that the plant nanovesicles are perfectly capable of retaining the drug without any kind of dispersion in the medium in which the nanovesicles are dispersed.

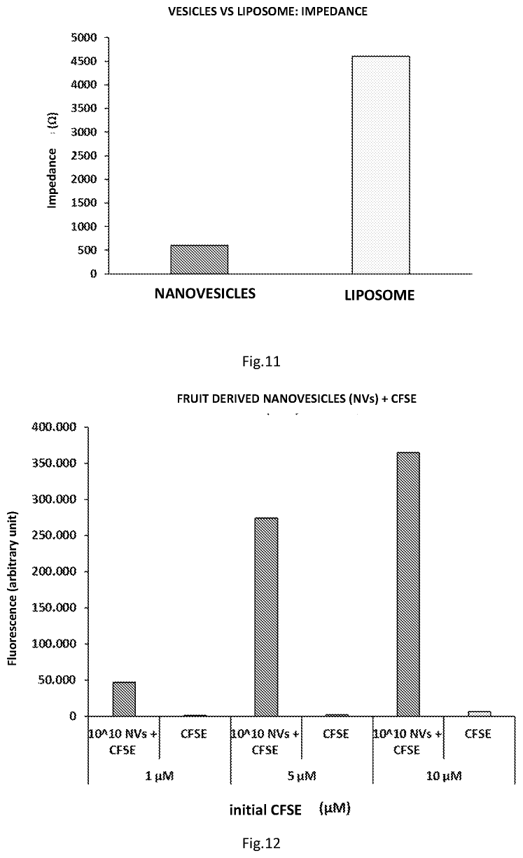

FIG. 11 shows a graph of the impedance of the nanovesicles extracted from organic lemon juice, which is significantly lower than the impedance recorded in liposomes. The impedance was measured following electroporation (300 V with a biphasic 1st pulse followed by a series of pulses, from 20V to 100 V) of the same number (106 to 1011) of nanovesicles and liposomes.

Advantageously, the impedance (W) obtained by means of the nanovesicle loading method for Acridine Orange (AO) was recorded.

Advantageously, different buffers with different ionic strengths were compared to evaluate the loading by the method of the present invention of compounds, such as Acridine Orange, into nanovesicles purified from juice extracted from organic fruits.

Stability tests of the retention of the loaded drug were carried out by electroporation within the plant-derived nanovesicles, left in 1×PBS These tests were conducted up to one month after electroporation by keeping the loaded nanovesicles at room temperature and at +4 C°. Stability tests were conducted by spectrofluorimeter under the same conditions as described above.

Advantageously, nanovesicles loaded with the desired compound were used for the in vitro treatment of tumor cells (e.g. Mel 501, CEM, CEM/VBL-100). The cytotoxic effect of the compound loaded into the nanovesicles was assessed after an incubation period of 1 h to 168 h under a light microscope using trypan blue assay. Transfer of the fluorescent compound to the cells was assessed by spectrofluorimeter (under the conditions described above) following resuspension of the cells in PBS (50 to 1000 ml). At the same time, the cells were also treated with the compound alone and loaded into the liposomes, making it possible to compare the cytotoxic and transfer effect of the compound when loaded into the nanovesicles.

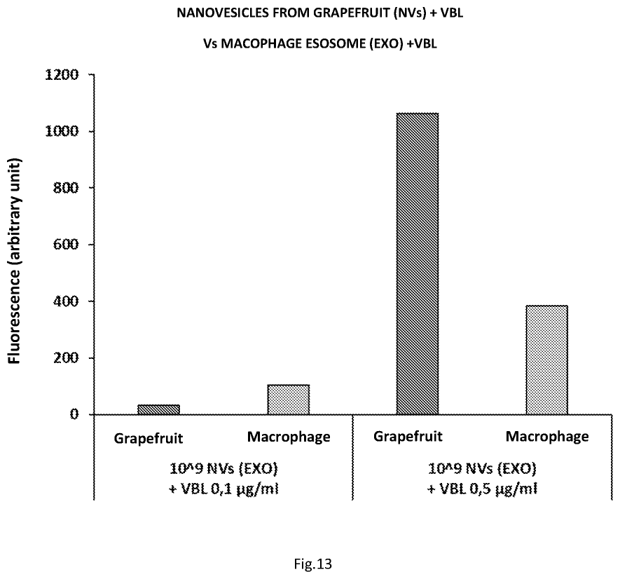

The same steps of the alternative passive diffusion method were used to load macrophage exosomes (10⁹) the compound (VBL) at concentrations from 0 to 0.5 pg/ml, to be used as a loading control. The tests performed on the nanovesicles treated according to the method are shown in FIGS. 12 and 13.

In particular, FIG. 12 shows loading tests, using the passive diffusion method of the present invention, of CFSE at increasing concentrations (0.5, 1 and 10 mM) in a fixed number of nanovesicles (NVs) (10¹⁰) isolated from a mix of organic fruit. The results depicted in FIG. 12 show that CFSE is loaded into plant nanovesicles by passive diffusion in a dose-dependent manner. Loading is confirmed by the negative control, CFSE alone, which does not emit any fluorescence.

Thus, CFSE crosses the membrane of the plant nanovesicles and binds covalently to all free amines on the surface and within the nanovesicles, emitting fluorescence.

FIG. 13 shows loading, by the passive diffusion method described in the present invention, of Vinblastine (VBL) into nanovesicles (NVs) isolated from biological pink grapefruit (Citrus paradisi) compared to loading into nanovesicles (exosomes, EXO) isolated from human macrophages. The same number of nanovesicles (from 109) isolated from biological pink grapefruit (see experimental protocol) and from human macrophages were loaded, by 2 h incubation at Tamb and shaking, with an initial concentration of 0.1 and 0.5 pg/mL of VBL. The results depicted in FIG. 13 show that VBL is loaded into the nanovesicles, by passive diffusion, in a dose-dependent manner.

VBL loading is relatively low at the lowest concentration (0.1 pg/ml) and is highest in macrophages; however, at the highest concentration of VBL (0.5 pg/ml) the loading capacity into the nanovesicles increases considerably resulting in significantly higher loading in grapefruit nanovesicles than in macrophage exosomes.

Although at least one exemplary embodiment has been presented in the summary and detailed description, it should be understood that there are a large number of variants falling within the scope of protection of the invention. Furthermore, it must be understood that the embodiment(s) presented are merely examples which are not intended to limit in any way the scope of protection of the invention or its application or configurations. Rather, the summary and detailed descriptions provide a convenient guide for the skilled person in the art to implement at least one exemplary embodiment, it being clear that numerous variations can be made in the function and assembly of the elements described herein, without exceeding the scope of protection of the invention as set forth in the appended claims and their technical-legal equivalents.

1. A method for loading various molecules into plant-derived nanovesicles, comprising the following steps:
a. suspending the isolated nanovesicles in a phosphate buffered saline;
b. analyzing the suspended nanovesicles with a technique called “Nanoparticle Tracking Analysis” using a “Nanosight” for the evaluation of concentration and size distribution;
c. re-suspending the nanovesicles in phosphate buffered saline;
d. transferring the nanovesicles to sterile means;
e. adding the fluorescent chemical to be loaded;
f. treating the nanovesicles in the sterile means to facilitate the entry of the molecule to be loaded through their cell membrane;
g. transferring the nanovesicles loaded with the desired molecule into ultra-centrifuge tubes;
h. re-suspending the pellet containing the nanovesicles in phosphate buffered saline and storing the supernatant obtained from the ultracentrifugation as a control for subsequent analysis;
i. re-suspending the nanovesicles in phosphate buffered saline and proceed with testing.

2. The method according to claim 1, wherein step f. is characterized by treating the nanovesicles with electroporation by means of an electroporator, using the following pulse trains:
300 V with eight pulses,
300 V with a first pulse,
a series of eight 20 V pulses, or 50 V pulses, or 80 V pulses, or 100 V pulses.

3. The method according to claim 2, wherein the sterile means of step d. are sterile cuvettes.

4. The method according to claim 1, wherein the nanovesicle transfer step d. is characterized by an amount of nanovesicles between 10⁶ and 10¹³.

5. The method according to claim 1, wherein the step e. of adding the fluorescent chemical compound to be loaded is characterized by a concentration between 0.1 to 100 pg/ml.

6. The method according to claim 1, wherein step f. is characterized by incubating the nanovesicles with the fluorescent chemical compound to be loaded, while stirring:
carboxyfluorescein succinimide ester (CFSE) for 30 min at 37° C., or
vinblastine (VBL) for 2 h at room temperature (T amb).

7. The method according to claim 1 wherein the sterile means of step d. are ultracentrifuge tubes.

8. The method according to claim 6 wherein the amount of nanovesicles in each ultracentrifugation tube is between 10⁹ and 10¹⁰.

9. The method according to claim 1, wherein the step e. of adding the chemical compound to be loaded is characterized by a concentration of between 1 and 10 mM carboxyfluorescein succinimide ester (CFSE) and a concentration of between 0.1 and 0.5 pg/mL vinblastine (VBL).

10. The method according to claim 1 wherein the chemical compounds to be made are vitamins, substances for cosmetic use, drugs and agents for medical, veterinary, pharmacological and food use.

11. The method according to claim 1 wherein the nanovesicles are obtained from at least one of the biological plants selected from
the group consisting of Citrus paradisi, Citrus Lemon (L), Citrus Reticulata, Citrus Bergamia, Actinidia Chinensis, Mangifera Indica, Carica Papaya Linn, Citrus Sinensis, Malus domestica.
